(19) United States
(12) Patent Application Publication
(10) Pub. No.: US 2013/0150719 A1
(43) Pub. Date: Jun. 13, 2013

(54) ULTRASOUND IMAGING SYSTEM AND METHOD

(71) Applicant: Fredrik Orderud, Oslo (NO)

(72) Inventor: Fredrik Orderud, Oslo (NO)

(73) Assignee: GENERAL ELECTRIC COMPANY, Schenectady, NY (US)

(21) Appl. No.: 13/314,599

(22) Filed: Dec. 8, 2011

(51) Int. Cl. A61B 8/13

(52) U.S. Cl. 600/443

(57) ABSTRACT

An ultrasound imaging system and method for ultrasound imaging. The method includes generating a volume-rendered image from three-dimensional ultrasound data. The volume-rendered image is colorized with at least two colors according to a depth-dependent color scheme. The method includes displaying the volume-rendered image. The method includes generating a planar image from the three-dimensional ultrasound data, where the planar image is colorized according to the same depth-dependent color scheme. The method includes displaying the planar image.

### FIELD OF THE INVENTION

This disclosure relates generally to an ultrasound imaging system and method for displaying a volume-rendered image and an planar image that are both colorized according to the same depth-dependent scheme.

### BACKGROUND OF THE INVENTION

Conventional ultrasound imaging systems acquire three-dimensional ultrasound data from a patient and are then able to generate and display multiple types of images from the three-dimensional ultrasound data. For example, conventional ultrasound imaging systems may generate and display a volume-rendered image based on the three-dimensional ultrasound data and/or conventional ultrasound imaging systems may generate one or more planar images from the three-dimensional ultrasound data. The volume-rendered image is a perspective view of surfaces rendered from the three-dimensional ultrasound data while the planar image is an image of a plane through the volume included within the three-dimensional ultrasound data. Users would typically use a volume-rendered image to get an overview of an organ or structure and then view one or more planar images of slices through the volume-rendered image in order to obtain more-detailed views of key portions of the patient's anatomy. Planar images generated from three-dimensional ultrasound data are very similar to images generated from conventional two-dimensional ultrasound modes, such as B-mode, where every pixel is assigned an intensity based on the amplitude of the ultrasound signal received from the location in the patient corresponding to the pixel.

Conventional ultrasound imaging systems typically allow the user to control rotation and translation of the volume-rendered image. In a similar manner, conventional ultrasound imaging systems allow the user to control the position of the plane being viewed in any planar images through adjustments in translation and tilt. Additionally, ultrasound imaging systems typically allow the user to zoom in on specific structures and potentially view multiple planar images, each showing a different plane through the volume captured in the three-dimensional ultrasound data. Due to all of the image manipulations that are possible on conventional ultrasound imaging systems, it is easy for users to become disoriented within the volume. Between adjustments and rotations to volume-rendered images and adjustments, including translations, rotations, and tilts to the planar images, it may be difficult for even an experienced clinician to remain oriented with respect to the patient's anatomy while manipulating and adjusting the volume-rendered image and/or the planar images.

For these and other reasons an improved method and system for generating and displaying images generated from three-dimensional ultrasound data is desired.

### BRIEF DESCRIPTION OF THE INVENTION

The above-mentioned shortcomings, disadvantages and problems are addressed herein which will be understood by reading and understanding the following specification.

In an embodiment, a method of ultrasound imaging includes generating a volume-rendered image from three-dimensional ultrasound data, wherein the volume-rendered image is colorized with at least two colors according to a depth-dependent color scheme. The method includes displaying the volume-rendered image. The method includes generating a planar image from the three-dimensional ultrasound data, wherein the planar image is colorized according to the same depth-dependent color scheme as the volume rendered image. The method also includes displaying the planar image.

In another embodiment, a method of ultrasound imaging includes generating a volume-rendered image from three-dimensional ultrasound data and applying a depth-dependent color scheme to the volume-rendered image. The method includes displaying the volume-rendered image after applying the depth-dependent color scheme to the volume-rendered image. The method includes generating a planar image of a plane that intersects the volume-rendered image, applying the depth-dependent color scheme to the planar image, and displaying the planar image after applying the depth-dependent color scheme to the planar image.

In another embodiment, an ultrasound imaging system includes a probe adapted to scan a volume of interest, a display device, a user interface, and a processor in electronic communication with the probe, the display device, and the user interface. The processor is configured to generate a volume-rendered image from the three-dimensional ultrasound data, apply a depth-dependent color scheme to the volume-rendered image, and display the volume-rendered image on the display device. The processor is configured to generate a planar image of a plane that intersects the volume-rendered image, apply the depth-dependent color scheme to the planar image, and display the planar image on the display device at the same time as the volume-rendered image.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram of an ultrasound imaging system in accordance with an embodiment;

FIG. 2 is a schematic representation of the geometry that may be used to generate a volume-rendered image in accordance with an embodiment;

FIG. 3 is a schematic representation of a screenshot in accordance with an embodiment; and

FIG. 4 is a flow chart showing the steps of a method in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

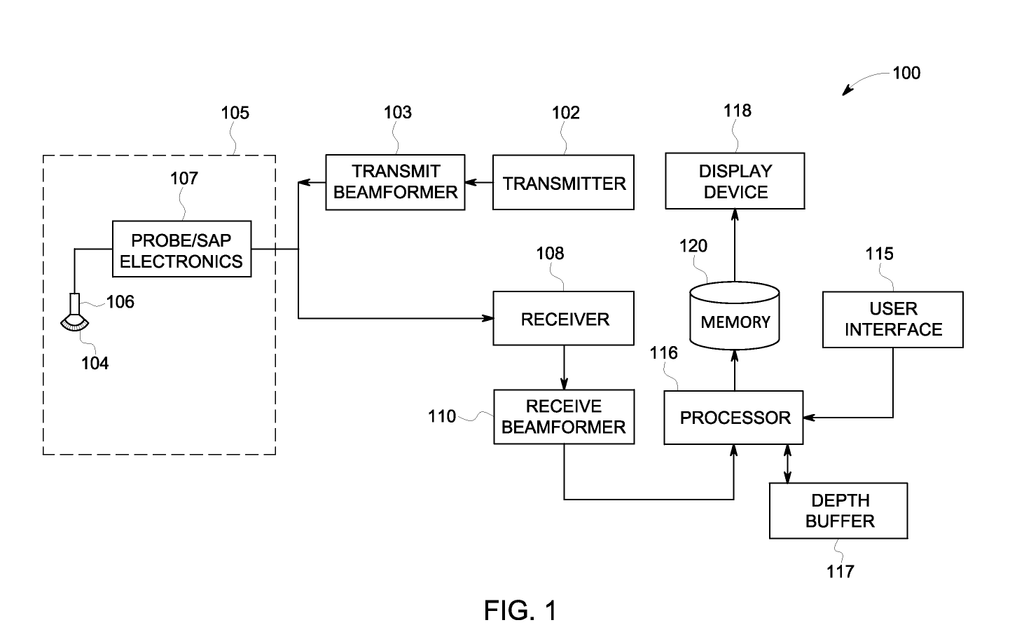

FIG. 1 is a schematic diagram of an ultrasound imaging system 100 in accordance with an embodiment. The ultrasound imaging system 100 includes a transmitter 102 that transmits a signal to a transmit beamformer 103 which in turn drives transducer elements 104 within a transducer array 106 to emit pulsed ultrasonic signals into a structure, such as a patient (not shown). A probe 105 includes the transducer array 106, the transducer elements 104 and probe/SAP electronics 107. The probe 105 may be an electronic 4D (E4D) probe, a mechanical 3D probe, or any other type of probe capable of acquiring three-dimensional ultrasound data. The probe/SAP electronics 107 may be used to control the switching of the transducer elements 104. The probe/SAP electronics 107 may also be used to group the transducer elements 104 into one or more sub-apertures. A variety of geometries of transducer arrays may be used. The pulsed ultrasonic signals are back-scattered from structures in the body, like blood cells or muscular tissue, to produce echoes that return to the transducer elements 104. The echoes are converted into electrical signals, or ultrasound data, by the transducer elements 104 and the electrical signals are received by a receiver 108. The electrical signals representing the received echoes are passed through a receive beam-former 110 that outputs ultrasound data or three-dimensional ultrasound data. A user interface 115 may be used to control operation of the ultrasound imaging system 100, including, to control the input of patient data, to change a scanning or display parameter, and the like.

The ultrasound imaging system 100 also includes a processor 116 to process the ultrasound data and generate frames or images for display on a display device 118. The processor 116 may include one or more separate processing components. For example, the processor 116 may include a central processing unit (CPU), a microprocessor, a graphics processing unit (GPU), or any other electronic component capable of processing inputted data according to specific logical instructions. Having a processor that includes a GPU may advantageous for computation-intensive operations, such as volume-rendering, which will be described in more detail hereinafter. The processor 116 is in electronic communication with the probe 105, the display device 118, and the user interface 115. The processor 116 may be hard-wired to the probe 105 and the display device 118, and the user interface 115, or the processor 116 may be in electronic communication through other techniques including wireless communication. The display device 118 may be a flat panel LED display according to an embodiment. The display device 118 may include a screen, a monitor, a projector, a flat panel LED, or a flat panel LCD according to other embodiments.

The processor 116 may be adapted to perform one or more processing operations according to a plurality of selectable ultrasound modalities on the ultrasound data. Other embodiments may use multiple processors to perform various processing tasks. The processor 116 may also be adapted to control the acquisition of ultrasound data with the probe 105. The ultrasound data may be processed in real-time during a scanning session as the echo signals are received. For purposes of this disclosure, the term “real-time” is defined to include a process performed with no intentional lag or delay. An embodiment may update the displayed ultrasound image at a rate of more than 20 times per second. The images may be displayed as part of a live image. For purposes of this disclosure, the term “live image” is defined to include a dynamic image that is updated as additional frames of ultrasound data are acquired. For example, ultrasound data may be acquired even as images are being generated based on previously acquired data and while a live image is being displayed. Then, according to an embodiment, as additional ultrasound data are acquired, additional frames or images generated from more-recently acquired ultrasound data are sequentially displayed. Additionally or alternatively, the ultrasound data may be stored temporarily in a buffer during a scanning session and processed in less than real-time in a live or off-line operation. Other embodiments of the invention may include multiple processors (not shown) to handle the processing tasks. For example, a first processor may be utilized to demodulate and decimate the ultrasound signal while a second processor may be used to further process the data prior to displaying an image. It should be appreciated that other embodiments may use a different arrangement of processors.

The processor 116 may be used to generate an image, such as a volume-rendered image or a planar image, from a three-dimensional ultrasound data acquired by the probe 105. According to an embodiment, the three-dimensional ultrasound data includes a plurality of voxels, or volume elements. Each of the voxels is assigned a value or intensity based on the acoustic properties of the tissue corresponding to a particular voxel.

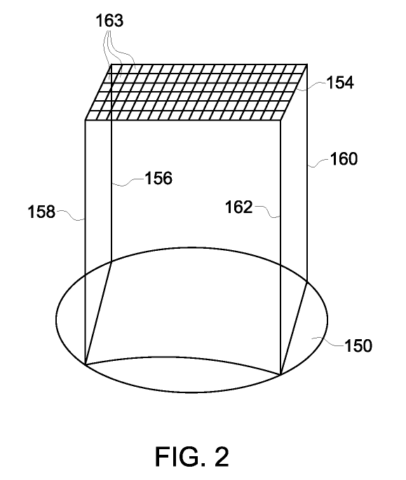

FIG. 2 is a schematic representation of the geometry that may be used to generate a volume-rendered image according to an embodiment. FIG. 2 includes a three-dimensional ultrasound dataset 150 and a view plane 154.

Referring to both FIGS. 1 and 2, the processor 116 may generate a volume-rendered image according to a number of different techniques. According to an exemplary embodiment, the processor 116 may generate a volume-rendered image through a ray-casting technique from the view plane 154. The processor 116 may cast a plurality of parallel rays from the view plane 154 to the three-dimensional ultrasound data 150. FIG. 2 shows ray 156, ray 158, ray 160, and ray 162 bounding the view plane 154. It should be appreciated that many more rays may be cast in order to assign values to all of the pixels 163 within the view plane 154. The three-dimensional ultrasound data 150 comprises voxel data, where each voxel is assigned a value or intensity. According to an embodiment, the processor 116 may use a standard “front-to-back” technique for volume composition in order to assign a value to each pixel in the view plane 154 that is intersected by a ray. Each voxel may be assigned a value and an opacity based on information in the three-dimensional ultrasound data 150. For example, starting at the front, that is the direction from which the image is viewed, each value along a ray may be multiplied with a corresponding opacity. This generates opacity-weighted values, which are then accumulated in a front-to-back direction along each of the rays. This process is repeated for each of the pixels 163 in the view plane 154 in order to generate a volume-rendered image. According to an embodiment, the pixel values from the view plane 154 may be displayed as the volume-rendered image. The volume-rendering algorithm may be configured to use an opacity function providing a gradual transition from opacities of zero (completely transparent) to 1.0 (completely opaque). The volume-rendering algorithm may factor the opacities of the voxels along each of the rays when assigning a value to each of the pixels 163 in the view plane 154. For example, voxels with opacities close to 1.0 will block most of the contributions from voxels further along the ray, while voxels with opacities closer to zero will allow most of the contributions from voxels further along the ray. Additionally, when visualizing a surface, a thresholding operation may be performed where the opacities of voxels are reassigned based on a threshold. According to an exemplary thresholding operation, the opacities of voxels with values about the threshold may be set to 1.0 while voxels with the opacities of voxels with values below the threshold may be set to zero. This type of thresholding eliminates the contributions of any voxels other than the first voxel above the threshold along the ray. Other types of thresholding schemes may also be used. For example, an opacity function may be used where voxels that are clearly above the threshold are set to 1.0 (which is opaque) and voxels that are clearly below the threshold are set to zero (translucent). However, an opacity function may be used to assign opacities other than zero and 1.0 to the voxels with values that are close to the threshold. This “transition zone” is used to reduce artifacts that may occur when using a simple binary thresholding algorithm. For example, a linear function mapping opacities to values may be used to assign opacities to voxels with values in the “transition zone”. Other types of functions that progress from zero to 1.0 may be used in accordance with other embodiments.

In an exemplary embodiment, gradient shading may be used to generate a volume-rendered image in order to present the user with a better perception of depth regarding the surfaces. For example, surfaces within the three-dimensional ultrasound data 150 may be defined partly through the use of a threshold that removes data below or above a threshold value. Next, gradients may be defined at the intersection of each ray and the surface. As described previously, a ray is traced from each of the pixels 163 in the view plane 154 to the surface defined in the dataset 150. Once a gradient is calculated at each of the rays, a processor 116 (shown in FIG. 1) may compute light reflection at positions on the surface corresponding to each of the pixels 163 and apply standard shading methods based on the gradients. According to another embodiment, the processor 116 identifies groups of connected voxels of similar intensities in order to define one or more surfaces from the 3D data. According to other embodiments, the rays may be cast from a single view point.

According to all of the non-limiting examples of generating a volume-rendered image listed hereinabove, the processor 116 may use color in order to convey depth information to the user. Still referring to FIG. 1, as part of the volume-rendering process, a depth buffer 117 may be populated by the processor 116. The depth buffer 117 contains a depth value assigned to each pixel in the volume-rendered image. The depth value represents the distance from the pixel to a surface within the volume shown in that particular pixel. A depth value may also be defined to include the distance to the first voxel that is a value above that of a threshold defining a surface. Each depth value may be associated with a color value according to a depth-dependent scheme. This way, the processor 116 may generate a volume-rendered image that is colorized according to a depth-dependent color scheme. For example, each pixel in the volume-rendered image may be colorized according to its depth from the view plane 154 (shown in FIG. 2). According to an exemplary colorization scheme, pixels representing surfaces at a first plurality of depths, such as structures at relatively shallow depths, may be depicted in a first color, such as bronze. Pixels representing surfaces at a second plurality of depths, such as deeper depths, may be depicted in a second color, such as blue. Varying intensities of the first color and the second color may be used to provide additional depth cues to the viewer. Additionally, the color used for the pixels may smoothly progress from bronze to blue with increasing depth according to an embodiment. It should be appreciated by those skilled in the art, that many other depth dependent color schemes, including those that use different colors, and/or more than two different colors, may be used in accordance with other embodiments.

Still referring to FIG. 1, the ultrasound imaging system 100 may continuously acquire ultrasound data at a frame rate of, for example, 5 Hz to 50 Hz depending on the size and spatial resolution of the ultrasound data. However, other embodiments may acquire ultrasound data at a different rate. A memory 120 is included for storing processed frames of acquired ultrasound data that are not scheduled to be displayed immediately. The frames of ultrasound data are stored in a manner to facilitate retrieval thereof according to the order or time of acquisition. As described hereinabove, the ultrasound data may be retrieved during the generation and display of a live image. The memory 120 may include any known data storage medium.

Optionally, embodiments of the present invention may be implemented utilizing contrast agents. Contrast imaging generates enhanced images of anatomical structures and blood flow in a body when using ultrasound contrast agents including microbubbles. After acquiring ultrasound data while using a contrast agent, the image analysis includes separating harmonic and linear components, enhancing the harmonic component and generating an ultrasound image by utilizing the enhanced harmonic component. Separation of harmonic components from the received signals is performed using suitable filters. The use of contrast agents for ultrasound imaging is well known by those skilled in the art and will therefore not be described in further detail.

In various embodiments of the present invention, ultrasound data may be processed by other or different mode-related modules. The images are stored and timing information indicating a time at which the image was acquired in memory may be recorded with each image. The modules may include, for example, a scan conversion module to perform scan conversion operations to convert the image frames from Polar to Cartesian coordinates. A video processor module may be provided that reads the images from a memory and displays the image in real time while a procedure is being carried out on a patient. A video processor module may store the image in an image memory, from which the images are read and displayed. The ultrasound imaging system 100 shown may be a console system, a cart-based system, or a portable system, such as a hand-held or laptop-style system according to various embodiments.

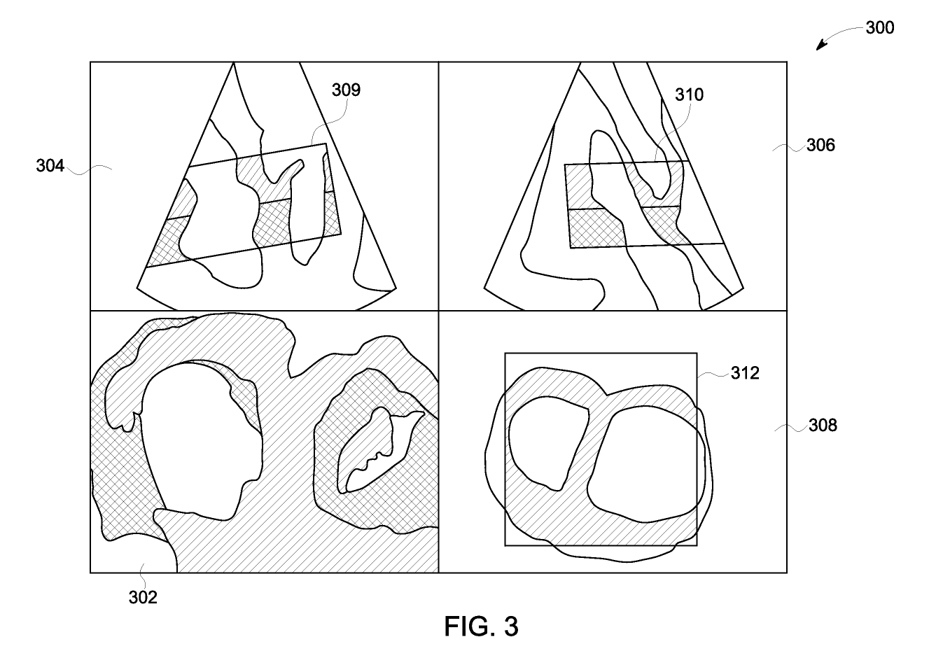

FIG. 3 is a schematic representation of a screen shot 300 that may be displayed in accordance with an embodiment. The screen shot 300 is divided into 4 regions in accordance with an exemplary embodiment. A separate image may be displayed in each of the regions. The screen shot 300 may be displayed on a display device such as the display device 118 shown in FIG. 1.

The screen shot 300 includes a volume-rendered image 302, a first planar image 304, a second planar image 306, and a third planar image 308. FIG. 3 will be described in additional detail hereinafter.

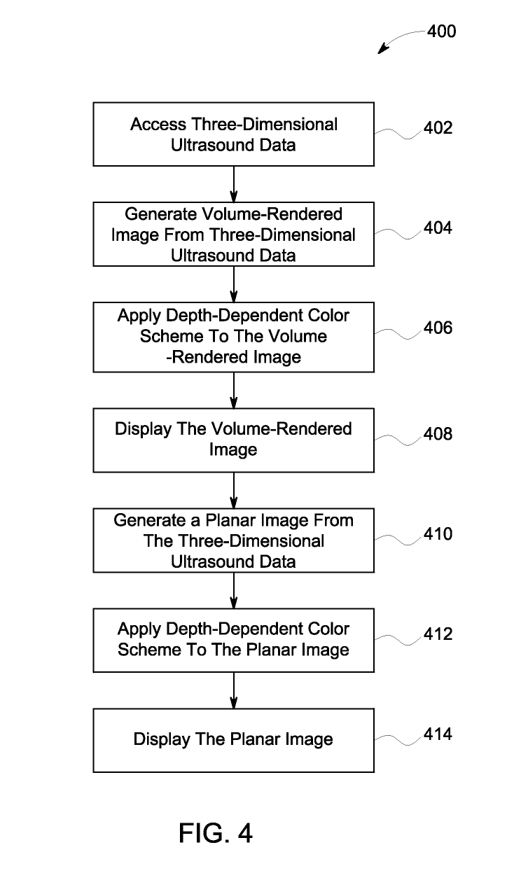

Referring to FIG. 4, a flow chart is shown in accordance with an embodiment. The individual blocks represent steps that may be performed in accordance with the method 400. Additional embodiments may perform the steps shown in a different sequence and/or additional embodiments may include additional steps not shown in FIG. 4. The technical effect of the method 400 is the display of a volume-rendered image that has been colorized according to a depth-dependent color scheme and the display of an planar image that has been colorized according to the same depth-dependent color scheme. The method 400 will be described according to an exemplary embodiment where the method is implemented by the processor 116 of the ultrasound imaging system 100 of FIG. 1. It should be appreciated by those skilled in the art that different ultrasound imaging systems may be used to implement the steps of the method 400 according to other embodiments. Additionally, according to other embodiments, the method 400 may be performed by a workstation that has access to three-dimensional ultrasound data that was acquired by a separate ultrasound imaging system.

Referring now to FIGS. 1, 3 and 4, at step 402 the processor 116 accesses three-dimensional ultrasound data. According to an embodiment, the three-dimensional ultrasound data may be accessed in in real-time as the data is acquired by the probe 105. According to other embodiment, the processor 116 may access the three-dimensional ultrasound data from a memory or storage device. At step 404, the processor 116 generates a volume-rendered image from the three-dimensional ultrasound data. At step 406, the processor 116 applies a depth dependent color scheme to the volume-rendered image in order to colorize the volume-rendered image. The processor 116 may colorize the pixels of the volume-rendered image based on the depths associated with each of the pixels. The depth information for each of the pixels may be located in the depth buffer 117. Therefore, the processor 116 may access the depth buffer 117 to determine the depths of the structures represented in each of the pixels. For example, pixels representing structures within a first range of depths from a view plane may be assigned a first color and pixels representing structures within a second range of depths may be assigned a second color that is different from the first color. If the structure represented by the pixel is within a first range of depths from the view plane, then the processor 116 may assign the first color to the pixel. On the other hand, if the structure represented by the pixel is within the second range of depths from the view plane, then the processor 116 may assign the second color to the pixel. According to an embodiment, the first range of depths may be shallower than the second range of depths.

At step 408 the processor 116 displays a volume-rendered image, such as volume-rendered image 302, on the display device 118. It should be noted that the volume-rendered image 302 is displayed after the processor 116 has applied the depth-dependent color scheme to the volume-rendered image at step 406. As such, the pixels in the volume-rendered image 302 are colorized according to the depths of the structure represented in each of the pixels. On FIG. 3, regions that are colored with a first color are represented with single hatching while regions that are colored with a second color are represented with cross-hatching. According to an exemplary embodiment, volume-rendered image 302 depicts a volume-rendering of a patient's heart. A mitral valve and a tricuspid valve are visible in the volume-rendered image 302. According to an embodiment, all of the regions colorized in the first color (depicted with single hatching) represent structures that are closer to a view plane, and hence closer to the viewer looking at the display device 118. Meanwhile, all of the regions colorized in the second color (depicted with cross-hatching) represent structures that are further from the view plane and the viewer. Colorizing a volume-rendered image according to a depth-dependent color scheme makes it easier for a viewer to interpret and understand the relative depths of structures represented in a volume-rendered image. Without some type of depth-dependent color scheme, it may be difficult for a viewer to determine if a structure shown in a volume-rendered image is at a deeper or a shallower depth than other structures depicted in the volume-rendered image.

Still referring to FIGS. 1, 3, and 4, at step 410, the processor 116 generates a planar image from the three-dimensional ultrasound data accessed during step 402. According to an embodiment, the planar image may be a four-chamber view of a heart, such as that shown in the first planar image 304 in FIG. 3. For the rest of the description, the method 400 will be described according to an exemplary embodiment where the planar image is the first planar image 304. It should be appreciated that according to other embodiments, the planar image may depict different planes. The first planar image 304 intersects the volume-rendered image 302.

Next, at step 412, the processor 116 applies the depth-dependent color scheme to a portion of the first planar image 304. The processor 116 colorizes the first planar image 304 by applying the same depth-dependent color scheme that was used to colorize the volume-rendered image 302. In other words, the same colors are associated with the same ranges of depths when colorizing both the volume-rendered image 302 and the first planar image 304. As with the volume-rendered image 302, the hatching and the cross-hatching represent the regions of the first planar image 304 that are colored the first color and the second color respectively. According to an embodiment, only the portions of the first planar image 304 within a first view port 309 are colored according to the depth-dependent color scheme. For example, the processor 116 may access the depth buffer 117 in order to determine the depths of the structures associated with each of the pixels in the first planar image. Then, the processor 116 may colorize the first planar image based on the same depth-dependent color scheme used to colorize the volume-rendered image. That is, the processor 116 may assign the same first color to pixels showing structures that are within the first range of depths and the processor 116 may assign the same second color to pixels showing structures within the second range of depths. The first view port 309 graphically shows the extent of the volume of data used to generate the volume-rendered image 302. In other words, the first view port 309 shows the intersection of the plane shown in the first planar image 304 and the volume from which the volume-rendered image 302 is generated. According to an embodiment, the user may manipulate the first view port 309 through the user interface 115 in order to alter the size and/or the shape of the data used to generate the volume-rendered image 302. For example, the user may use a mouse or trackball of the user interface 115 to move a corner or a line of the first view port 309 in order to change the size and/or the shape of the volume used to generate the volume-rendered image 302. According to an embodiment, the processor 116 may generate and display an updated volume-rendered image in response to the change in volume size or shape as indicated by the adjustment of the first view port 309. The updated volume-rendered image may be displayed in place of the volume-rendered image 302. For example, if the user were to change the first view port 309 so that the first view port 309 was smaller in size, then the volume-rendered image would be regenerated using a smaller volume of data. Likewise, if the user were to change the first view port 309 so that the first view port 309 was larger in size, an updated volume-rendered image would be generated based on a larger volume of data. According to an embodiment, updated volume-rendered images may be generated and displayed in real-time as the user adjusts the first view port 309. This allows the user to quickly see the changes to the volume-rendered image resulting from adjustments in the first view port 309. The size and resolution of the three-dimensional ultrasound dataset used to generate the volume-rendered image as well as the speed of the processor 116 will determine how fast it is possible to generate and display the updated volume-rendered image. The updated volume-rendered image may be colorized according to the same depth-dependent color scheme as the volume-rendered image 302 and the first planar image 304.

Since the first planar image 304 is colorized according to the same depth-dependent color scheme as the volume-rendered image 302, it is very easy for a user to understand the precise location of structures located in the first planar image 304. For example, since structures represented in the first color (represented by the single hatching on FIG. 3) are closer to the view plane than structures represented in the second color (represented by the cross-hatching on FIG. 3), the user can easily see the position of the first planar image 304 with respect to the volume-rendered image 302. For example, the first planar image 304 includes both the first color (hatching) and the second color (cross-hatching) within the first view port 309. These colors are the same as the colors used within the volume-rendered image 302. As such, by looking at the colors in the first planar image 304, it is possible for the user to quickly and accurately determine the orientation of the plane represented in the first planar image 304 with respect to the volume-rendered image 302. Additionally, by viewing both the first planar image 304 and the volume-rendered image 302 at the same time, the user may rely on color to help positively identify one or more key structures within either of the images.

At step 414, the planar image is displayed. The planar image may include the first planar image 304. According to an exemplary embodiment, the first planar image 304 may be displayed on the display device 118 at the same time as the volume-rendered image as depicted in FIG. 3.

FIG. 3 includes the second planar image 306 and the third planar image 308 as well. According to an embodiment, the second planar image 306 and the third planar image 308 may be generated by iteratively repeating steps 410, 412, and 414 of the method 400 for each of the different planes. The second planar image includes a second view port 310 and the third planar image includes a third view port 312. According to an embodiment, the second planar image 306 may be a long-axis view, and the third planar image 308 may be a short axis view. The four-chamber view shown in the first planar image 304, the long-axis view, and the short axis view are all standard views used in cardiovascular ultrasound. However, it should be appreciated by those skilled in the art that other views may be used according to other embodiments. Additionally, other embodiments may display a different number of planar images at a time. For example, some embodiments may show more than three planar images, while other embodiments may show less than three planar images. Additionally, the number of planar images displayed at a time may be a user-selectable feature. The user may select the number planar images and the orientation of the planes according to an embodiment. According to an embodiment, the user may manipulate the second view port 310 and the third view port 312 in the same manner as that which was previously described with respect to the first view port 309. For example, the second view port 310 and the third view port 312 may indicate the portion of the data used to generate the volume-rendered image 302. The user may adjust the position of either the second view port 310 or the third view port 312 in order to alter the portion of the three dimensional ultrasound data used to generate the volume-rendered image 302. Additionally, it should be noted that according to an embodiment, the portions of the images within the viewports (309, 310, 312) are all colorized according to the same depth-dependent color scheme used to colorize the volume-rendered image. According to other embodiments, all of the first planar image 304, all of the second planar image 306, and all of the third planar image 308 may be colorized according to the same depth-dependent color scheme.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound imaging comprising:
generating a volume-rendered image from three-dimensional ultrasound data, wherein the volume-rendered image is colorized with at least two colors according to a depth-dependent color scheme;
displaying the volume-rendered image;
generating a planar image from the three-dimensional ultrasound data,
wherein the planar image is colorized according to the same depth-dependent color scheme as the volume-rendered image; and
displaying the planar image.

2. The method of claim 1, wherein the depth-dependent color scheme comprises a first color assigned to pixels representing structures at a first plurality of depths and a second color assigned to pixels representing structures at a second plurality of depths.

3. The method of claim 1, wherein the planar image comprises an image of a plane that intersects the volume-rendered image.

4. The method of claim 1, wherein the planar image and the volume-rendered image are both displayed at the same time.

5. The method of claim 4, further comprising displaying a view port on the planar image, wherein the view port at least partially defines the volume used to generate the volume-rendered image.

6. The method of claim 4, wherein the planar image is colorized according to the depth-dependent color scheme only within the view port.

7. The method of claim 5, further comprising adjusting the shape of the view port through a user interface.

8. The method of claim 7, further comprising generating and displaying an updated volume-rendered image in real-time after said adjusting the shape of the view port, wherein the ultrasound data used to generate the updated volume-rendered image is at least partially defined by the view port.

9. The method of claim 1, further comprising generating a second planar image that is colorized according to the depth-dependent color scheme.

10. The method of claim 9, further comprising displaying the second planar image at the same time as the planar image and the volume-rendered image.

11. A method of ultrasound imaging comprising:
generating a volume-rendered image from three-dimensional ultrasound data;
applying a depth-dependent color scheme to the volume-rendered image;
displaying the volume-rendered image after applying the depth-dependent color scheme to the volume-rendered image;
generating an planar image of a plane that intersects the volume-rendered image;
applying the depth-dependent color scheme to the planar image; and
displaying the planar image after applying the depth-dependent color scheme to the planar image.

12. The method of claim 11, wherein the depth-dependent color scheme comprises a first color assigned to pixels representing structures that are closer to a view plane and a second color assigned to pixels representing structures that are further from the view plane.

13. The method of claim 11, wherein the planar image and the volume-rendered image are displayed at the same time on a display device.

14. An ultrasound imaging system comprising:
a probe adapted to scan a volume of interest;
a display device;
a user interface; and
a processor in electronic communication with the probe, the display device and the user interface, wherein the processor is configured to:
generate a volume-rendered image from three-dimensional ultrasound data;
apply a depth-dependent color scheme to the volume-rendered image;
display the volume-rendered image on the display device;
generate an planar image of a plane that intersects the volume-rendered image;
apply the depth-dependent color scheme to the planar image; and
display the planar image on the display device at the same time as the volume-rendered image.

15. The ultrasound imaging system of claim 14, wherein the processor is further configured to display a view port on the planar image, wherein the view port at least partially defines the volume used to generate the volume-rendered image.

16. The ultrasound imaging system of claim 15, wherein the processor is further configured to generate an updated volume-rendered image in response to having a user adjust the view port.

17. The ultrasound imaging system of claim 16, wherein the processor is further configured to display the updated volume-rendered image on the display device in response to having the user adjust the position of the view port.

18. The ultrasound imaging system of claim 17, wherein the processor is further configured to display the updated volume-rendered image on the display device in real-time after the user adjusts the view port.

19. The ultrasound imaging system of claim 14, wherein the processor is further configured to generate a second planar image, wherein the second planar image comprises a second image of a second plane that is different from the plane.

20. The ultrasound imaging system of claim 14, wherein the processor is further configured to control the probe to acquire three-dimensional ultrasound data.
